(19) United States
(12) Patent Application Publication
(10) Pub. No.: US 2025/0032020 A1
(43) Pub. Date: Jan. 30, 2025

(54) BRAIN ACTIVITY STATE DETERMINATION DEVICE AND BRAIN ACTIVITY STATE DETERMINATION PROGRAM

(71) Applicants: TOSHIBA INFORMATION SYSTEMS (JAPAN) CORPORATION, Kawasaki-shi Kanagawa (JP); KYOTO UNIVERSITY, Kyoto-shi Kyoto (JP)

(72) Inventors: Hidetoshi OKUTOMI, Kawasaki-shi Kanagawa (JP); Tomoyuki MAO, Kawasaki-shi Kanagawa (JP); Ken UMENO, Kyoto-shi Kyoto (JP)

(73) Assignees: TOSHIBA INFORMATION SYSTEMS (JAPAN) CORPORATION, Kawasaki-shi Kanagawa (JP); KYOTO UNIVERSITY, Kyoto-shi Kyoto (JP)

(21) Appl. No.: 18/551,440

(22) PCT Filed: Mar. 29, 2023
(86) PCT No.: PCT/JP2023/013023

§ 371 Date: Sep. 20, 2023

(30) Foreign Application Priority Data
Mar. 31, 2022 (JP) 2022-061215

(51) Int. Cl. A61B 5/16; A61B 5/00; A61B 5/352

(52) U.S. Cl. CPC A61B 5/165; A61B 5/352; A61B 5/4064; A61B 5/7264; A61B 5/7275

(57) ABSTRACT

A brain activity determination device is equipped with a chaos index value calculation unit 201 for calculating a chaos index value, which is an index for determining the chaotic nature in chronologically ordered data; and a determination unit 205, which stores outputs obtained by inputting the RRI data obtained from a subject placed in a first state where the load on the brain is for obtaining reference value data, calculates an index value ratio that is a ratio of the reference value data and the chaos index value to be evaluated by inputting the RRI data obtained from a determination subject whose brain load is in a second state where evaluation target data is obtained to the chaos index value calculation unit 201 after obtaining the chaos index value to be evaluated, which is the data to be evaluated; and determines the brain activity state of the determination subject based on a comparison between the brain activity threshold and the index value ratio to determine the brain activity state.

### CROSS-REFERENCE TO RELATED APPLICATIONS

This application is a U.S. National Stage Application filed under 35 U.S.C. § 371 of International Application No. PCT/JP2023/013023, filed Mar. 29, 2023, which claims the benefit of priority to Japanese Patent Application No. JP 2022-061215 filed Mar. 31, 2022, which are hereby incorporated by reference in their entirety.

### TECHNICAL FIELD

This disclosure relates to a brain activity state determination device and a brain activity state determination program.

### BACKGROUND TECHNOLOGY

Conventionally, the state of brain activity is measured and determined by an electroencephalograph, which requires a large-sized apparatus and requires time and expense to measure.

For example, Patent Literature 1 discloses a brain activity measurement system comprising a brain activity measuring electrode 5 having multiple portions for acquiring electrical information by contacting the scalp, multiple guide bodies arranged around the scalp-contacting portions, a wet electrode mounting part, and the installation of a wet material that can be attached to and detached from the wet electrode mounting part. It may be used as a wet electrode when the wet material is attached to the wet electrode mounting part and can also be used as a dry electrode when the wet material is removed from the wet electrode mounting part.

This brain activity measurement system measures brain activities based on signals obtained by the measurement electrodes. When used with a means for securing the electrodes, such as an EEG cap or headset, the device can contact the scalp regardless of the subject's head shape and allows measurement when it is challenging to measure with a dry type.

Patent Literature 2 discloses a brain activity monitoring device that consists of sensors 20 that are attached to the subject's head and collect first and second data using near-infrared spectroscopy (NIRS). Specifically, the sensor 20 has a light source that emits near-infrared light with a wavelength of about 700 nm to about 900 nm, and a light-receiving sensor that contacts the subject's head.

The brain activity monitoring device indicates the activity state of the subject's brain, with reference to first data collected during a first period. It is equipped with a data acquisition unit that acquires second data collected during a second period following the first period and a center-of-gravity calculator that calculates the centroid of the first data on a phase plane where the Mahalanobis distance is defined. It is further equipped with a distance calculation unit that calculates a Mahalanobis distance from the center of gravity of the second data and calculates a temporal change in the Mahalanobis distance of the second data; a determination unit that determines whether or not the Mahalanobis distance of the second data exceeds a predetermined threshold more than a predetermined number of times; and an output unit for outputting information indicating the activity state of the subject's brain when it is determined that the Mahalanobis distance of the second data exceeds the predetermined threshold more than the predetermined number of times.

Patent Literature 3 discloses a method for initiating brain activities. This method prompts the user to perform cognitive function training at an appropriate time after aerobic exercise.

In this method the computer acquires measured values of vital data of the user wearing a device that measures predetermined vital data from the via the communication part and encourages the user to initiate aerobic exercise that improves their physical function. Then, it measures the amount of time the user has performed aerobic exercise, based on the vital data measurements, and displays a prompt for the user to end the aerobic exercise when the aerobic exercise exceeds a predetermined time. In addition, when a predetermined condition based on the measured value of vital data or the elapsed time from the end of the aerobic exercise is satisfied, it displays a prompt to perform predetermined cognitive function training to improve the user's brain function. The operation unit thus recognizes an operation by the user while the user is performing cognitive function training.

Since there is a relationship between heart rate and brain activities, the inventors of the present application proposed estimating drowsiness based on R-R Interval (RRI) data (Patent Literature 4).

Patent Literature 5 discloses a system configured to determine sleep stages of a subject based on cardiac artifact information and brain activity information in EEG signals. This system is based on a concern that cardiac artifacts present in EEG signals can occasionally trigger false sleep stage determination, which can lead to untimely sensory stimulation during sleep, the absence of stimulation, long periods of discarded EEG signal information, and/or other events. This system, compared to other existing technologies, improves real-time sleep staging and provides other benefits.

### BACKGROUND ART

### Patents

[Patent Literature 1] JP 2020-195777 A
[Patent Literature 2] JP 2020-130336 A
[Patent Literature 3] JP 2020-58725 A
[Patent Literature 4] JP 2018-57450 A
[Patent Literature 5] JP 2019-503746 A

### OVERVIEW

### Problem

As described above, in the conventional methods for observing brain activities, various devices have been devised, but current methods have not been developed sufficiently from the viewpoint of simplicity and accuracy. Therefore, the objective of this disclosure is to provide a brain activity determination device and a brain activity determination program that enable simpler and more accurate determination than existing methods.

### Methods for Solving the Problem

A brain activity determination device according to an embodiment of the present disclosure comprises a chaos index value calculation unit that calculates chaos index value, which is an index for determine the chaotic nature of the data in chronological order; a reference value data retention control unit that retains the output obtained by inputting the RRI data obtained from a subject placed in a first state, which is the state for obtaining the reference data, to the chaos index value calculation unit as reference value data in a storage device; a chaos index value calculation unit for obtaining the chaos index value to be evaluated, which is the data to be evaluated, by inputting the RRI data obtained from the subject to be evaluated whose brain load is considered to be in a second state in which evaluation target data is to be obtained, to the determination target chaos index value calculation unit; an index value ratio calculation unit for calculating the index value ratio, which is the ratio between the reference value data and the chaos index value to be evaluated; and determination unit for determining the brain activity state of the subject to be evaluated based on a comparison of the brain activity threshold and the index value ratio to determine a brain activity state.

A brain activity state determination program according to an embodiment of the present disclosure is characterized by programming a computer to function as: a chaos index value calculation unit that calculates chaos index value, which in index for determine the chaotic nature of the data in chronological order; a reference value data retention control unit that retains the output obtained by inputting the RRI data obtained from a subject placed in a first state, which is the state for obtaining the reference data, to the chaos index value calculation unit as reference value data in a storage device; a chaos index value calculation unit for obtaining the chaos index value to be evaluated, which is the data to be evaluated, by inputting the RRI data obtained from the subject to be evaluated whose brain load is considered to be in a second state in which evaluation target data is to be obtained, to the determination target chaos index value calculation unit; an index value ratio calculation unit for calculating the index value ratio, which is the ratio between the reference value data and the chaos index value to be evaluated; and determination unit for determining the brain activity state of the subject to be evaluated based on a comparison of the brain activity threshold and the index value ratio to determine a brain activity state.

### BRIEF DESCRIPTIONS OF THE DRAWINGS

FIG. 1 shows a histogram of the chaos index value ratio γ used in the brain activity determination apparatus according to an embodiment of the present disclosure when the subject is measured under Condition 1.

FIG. 2 shows a histogram of the chaos index value ratio γ used in the brain activity determination apparatus according to an embodiment of the present disclosure when the subject is measured under Condition 2.

FIG. 3 shows a device configuration diagram of the first embodiment of the brain activity determination device according to the present disclosure.

FIG. 4 is a diagram showing the operation up to storing resting chaos index value reference data CCI[1]-CCI[m] in an embodiment of the brain activity determination device using resting state reference data according to the present disclosure.

FIG. 5A is a diagram showing the operation up to storing resting chaos index value reference data Ref_{R}[1]-Ref_{R}[m] in an embodiment of the brain activity determination device using resting state reference data according to the present disclosure.

FIG. 5B is a diagram showing the operation up to obtaining the average index value ratio A V_{yA} in an embodiment of the brain activity determination device using resting state reference data according to the present disclosure.

FIG. 6 is a configuration diagram of the second embodiment of a brain activity determination device according to the present disclosure.

FIG. 7 shows a configuration diagram of a third embodiment of a brain activity determination device according to the present disclosure.

FIG. 8 shows a configuration diagram of a fourth embodiment of a brain activity determination device according to the present disclosure.

FIG. 9 is a diagram showing the operation up to storing resting chaos index value reference data CCI[1]-CCI[m] in an embodiment of the brain activity determination device using cognitive activity reference data according to the present disclosure.

FIG. 10A is a diagram showing the operation up to storing resting chaos index value reference data Ref_{R}[1]-Ref_{R}[m] in an embodiment of the brain activity determination device using cognitive activity reference data according to the present disclosure.

FIG. 10B is a diagram showing the operation up to obtaining the average index value ratio A V_{yA} in an embodiment of the brain activity determination device using cognitive activity reference data according to the present disclosure.

FIG. 11 is a diagram showing an example of long term processing results of using the embodiment using cognitive activity reference data by the brain activity state determination apparatus according to the present disclosure.

### DETAILED DESCRIPTION

A brain activity determination device and a brain activity determination program according to embodiments of the present disclosure will be described below with reference to the accompanying figures. In each figure, the same components are denoted by the same reference numerals, and overlapping descriptions are omitted. Embodiments of the present disclosure use a chaos index calculated from RRI data (heartbeat interval data).

The chaos index is an index for determining the chaotic nature of chronological data, and some chaos indices are listed below. Indices and references (other than (5) and (6)) are described individually below. Indices (7)-(9) are typical Lyapunov exponent estimation methods.
(1) ApEn (Approximate Entropy) [1] [2] [3] [4]
(2) SampEn (Sample Entropy) [3] [4]
(3) Fractal Dimension [5] [6]
(4) SD1/SD2 [7] [8]
(5) CD [Chaos Degree] JP 2018-120488 A
(6) ICD [Modified Chaos Degree] JP 2021-064323
(7) Estimation method of the Lyapunov exponent (Rosenstein's method) [9]
(8) Estimation method of the Lyapunov exponent (Wolf's method) [10]
(9) Estimation method of the Lyapunov exponent (Sano-Sawada's method) [11]

### REFERENCES

[1] Pincus, S. M., “Approximate entropy as a measure of system complexity,” PNAS 88, 2297-2301 (1991).
[2] Pincus, S. M., Gladstone, I. M. & Ehrenkranz, R. A, “A regularity statistic for medical data analysis,” J. Clin. Monit. Comput., 7, 335-345 (1991).
[3] Richman, J. S. & Moorman, J. R., “Physiological time-series analysis using approximate entropy and sample entropy,” Am. J. Physiol. Heart Circ. Physiol., 278, 2039-2049 (2000).
[4] Delgado-Bonal, A. & Alexander, M., “Approximate Entropy and Sample Entropy: A Comprehensive Tutorial,” Entropy, 21, 541 (2019).
[5] Higuchi, T., “Approach to an irregular time series on the basis of the fractal theory,” Physica D., 31, 277-83 (1998).
[6] Ahammer, H., “Higuchi dimension of digital images,” PLoS One, 6, e0119394 (2011).
[7] Hoshi, R. A., Pastre, C. M., Vanderlei, L. M., & Godoy, M. F., “Poincare plot indexes of heart rate variability: Relationships with other nonlinear variables,” Auton. Neurosci., 177, 271-274 (2013).
[8] Guzik, P. et. al., “Correlations between the Poincare plot and conventional heart rate variability parameters assessed during paced breathing,” J Physiol. Sci., 57, 63-71 (2007).
[9] Michael T. Rosenstein, James J. Collins, & Carlo J. De Luca, “A practical method for calculating largest Lyapunov exponents from small data sets,” Physica D: Nonlinear Phenomena, 65, 117-134 (1993).
[10] Alan Wolf, Jack B. Swift, Harry L. Swinney, & John A. Vastano, “Determining Lyapunov exponents from a time series,” Physica D: Nonlinear Phenomena, 16, 285-317 (1985).
[11] Sato, Shinichi, Sano, Masaki, & Sawada, Yasuji, “Practical Methods of Measuring the Generalized Dimension and the Largest Lyapunov Exponent in High Dimensional Chaotic Systems,” Theor. Phys., 77 (1987).

The inventors of the present application conducted an experiment to confirm that it is appropriate to use chaos index values obtained from RRI data to determine the state of brain activity. This experiment was performed on 18 healthy participants. Thirteen of the participants were in their 20s, two in their 30s, and three in their 50s; 15 of the participants were males and three were females. This experiment was conducted with the approval of the Kyoto University Graduate School of Informatics Research Ethics Committee (Approval Number: KUIS-EAR-2019-006).

Participants wore a Polar H10 chest strap heart rate sensor capable of measuring RRI and performed two experiments measuring RRI in the following conditions.
Rest: Sit in a chair and rest. No physical or mental strain.
Standing: Maintain an upright posture. Only physical load is applied.
Brain Task: Sit in a chair and perform a cognitive task (mental arithmetic or Sudoku). Only mental load is applied.

In Experiment 1, mental arithmetic was used as a brain task. The RRI data of the participants were measured for seven minutes at rest (denoted as Rest 1), seven minutes while standing (denoted as Standing), and seven minutes for mental arithmetic (denoted as Brain Task 1). A five-minute break was provided between each state. Participants repeated this experiment five times.

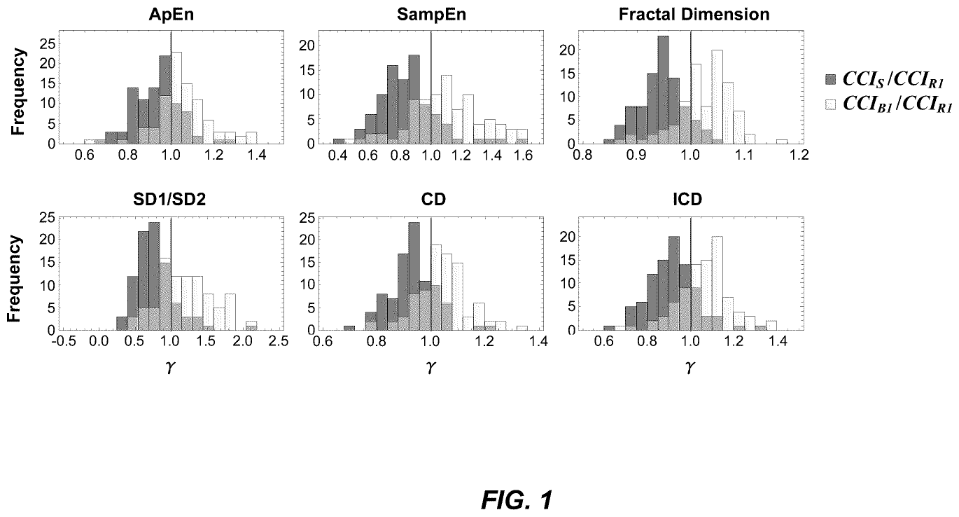

In each case, the chaos index value ratio γ (CCI_{S}/CCI_{R1}) is obtained where the chaos index value CCI_{R1} obtained using the RRI data from the resting state is the denominator, and the chaos index value CCI_{S} obtained using the RRI data from the standing state is the numerator. After that, the subjects are shifted to the brain task state, and the RRI data are measured in the brain task state. In the brain task state, subjects sat on a chair and performed mental arithmetic (single-digit addition) on a table. In this case, the chaos index value ratio γ (CCI_{B1}/CCI_{R1}) is obtained where the chaos index value CCI_{R1} obtained using the RRI data from the resting state is the denominator, and the chaos index value CCI_{B1} obtained using the RRI data from the brain task state is the numerator. FIG. 1 shows a grayscale histogram in which the frequency of the chaos index value ratio γ (CCI_{S}/CCI_{R1}) is indicated in dark gray and the frequency of the chaos index value γ (CCI_{B1}/CCI_{R1}) is indicated in light gray. Six types of chaos indices, ApEn, SampEn, Fractal Dimension, SD1/SD2, CD, and ICD, were used as illustrated.

In Experiment 2, Sudoku was used as a brain task. Participants measured the RRI for seven minutes at rest (denoted as Rest2) and for seven minutes during Sudoku (denoted as Brain Task2). A five-minute rest period was provided between each state. Participants repeated this experiment five times.

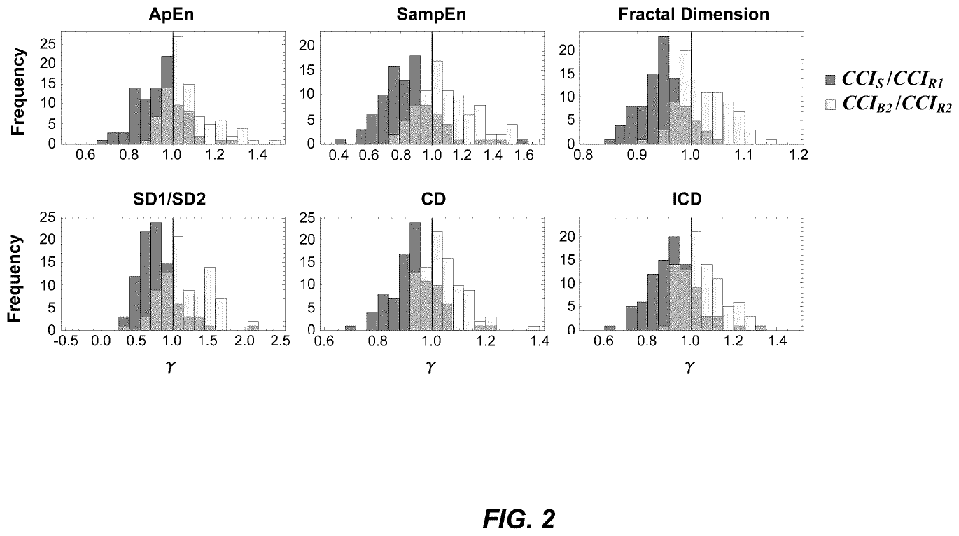

In this case, the chaos index value ratio γ (CCI_{B2}/CCI_{R2}) is obtained where CCI_{R2} obtained using the RRI data at rest is used as the denominator, and the chaos index value CCI_{B2} obtained using the RRI data from the brain task state is used as the numerator. FIG. 2 shows a grayscale histogram in which the frequency of the chaos index value ratio γ (CCI_{S}/CCI_{R1}) is indicated in dark gray, and the frequency of the chaos index value ratio γ (CCI_{B2}/CCI_{R2}) is indicated in light gray. Six types of chaos indices, ApEn, SampEn, Fractal Dimension, SD1/SD2, CD, and ICD, were used as illustrated.

In FIGS. 1 and 2, γ in the brain task state (light gray) is larger than γ in the standing state (dark gray) when any of the chaos indices is used, and γ is roughly 1 at the boundary of the standing state (dark gray) and the brain task state (light gray).

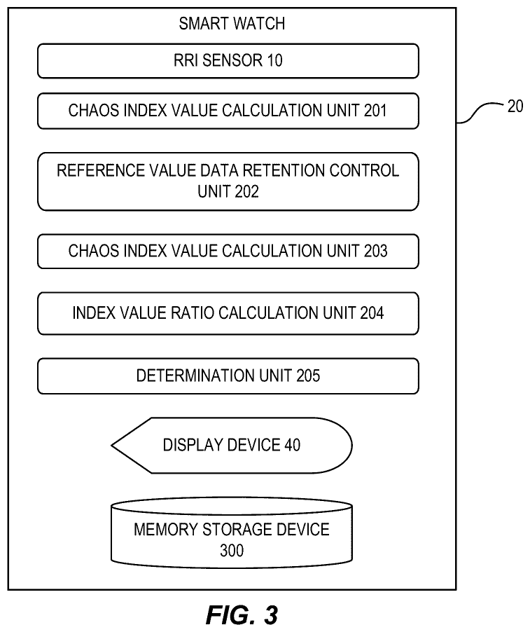

FIG. 3 shows a device configuration diagram of a first embodiment of a brain activity state determination device according to the present disclosure. In this embodiment, a smartwatch 20 is provided with all the components of the brain activity determination device. This smartwatch 20 has an RRI sensor 10 that detects a signal corresponding to the R wave of an electrocardiogram signal. A heart rate sensor can be used as this RRI sensor 10.

The RRI sensor 10 can be the part of the configuration of an ECG that extracts the electrocardiogram signals or pulse wave sensor, in addition to the heart rate sensor. The brain activity determination device may have a configuration other than the smartwatch 20. For example, the RRI sensor 10 may be provided in a living body, detect an electrocardiogram signal wirelessly or by wire, and output an RRI signal (before plastic surgery).

The smartwatch 20 may be configured as a computer and include chaos index value calculation unit 201, reference value data retention control unit 202, determination target chaos index value calculation unit 203, index value ratio calculation unit 204, and determination unit 205 and a storage device 300, which are realized as in a computer. Note that the storage device 300 may exist in the cloud, and the smartwatch 20, or computer, may communicate with the storage device 300 to transmit and receive data.

The chaos index value calculation unit 201 calculates a chaos index value, which is an index for determining the chaotic nature in a chronologically ordered data. The chaos index value calculation unit 201 calculates one or more of chaos index values. In terms of the types of chaos index in this case, the nine types mentioned above, or those in which similar indices are added can be employed. The reference value data retention control unit 202 inputs the RRI data obtained from the subject whose load on the brain is in the first state in which the reference value data is obtained by the RRI sensor 10 to the chaos index value calculation unit 201. Then, the obtained output is stored in the storage device 300 as reference value data.

The determination target chaos index value calculation unit 203 inputs RRI data obtained from the determination target person in the second state, which is a state in which load evaluation target data for the brain is obtained, to the chaos index value calculation unit 201. It then determines the chaos index value to be evaluated. The index value ratio calculation unit 204 calculates an index value ratio, which is a ratio between the reference value data and the chaos index value to be evaluated. The determination unit 205 determines the brain activity state of the person under evaluation based on a comparison between the brain activity threshold and the index value ratio to determine the brain activity state.

Since the chaos index value calculation unit 201 calculates one or more kinds of chaos index values, the above mentioned reference value data retention control unit 202 can retain the reference value data corresponding to the plurality of types in the storage device. The determination target chaos index value calculation unit 203 obtains determination target chaos index values corresponding to the plurality of types, and the index value ratio calculation unit 204 calculates index value ratios corresponding to the plurality of types. The index value ratio calculation unit 204 calculates the index value ratios corresponding to the plurality of types and averages the obtained index value ratios to obtain an average index value ratio.

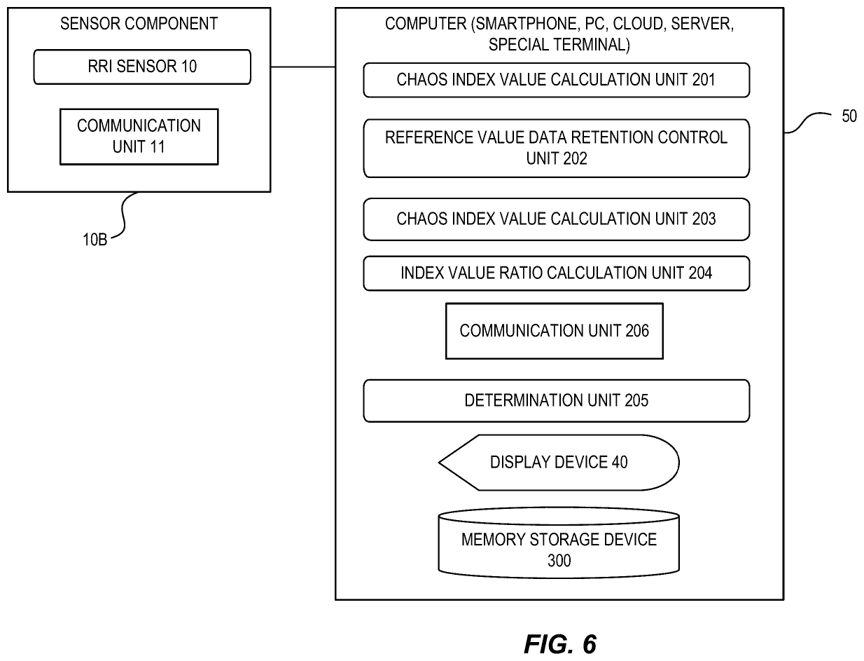

FIG. 6 shows a device configuration diagram of the second embodiment of the brain activity state determination device according to the present disclosure. In this embodiment, the brain activity state determination device can be composed of, for example, a sensor section 10B, which may be a disk-shaped housing attached to the body of the determination subject, and the computer 50. The computer 50 refers to a device such as a smartphone, personal computer, cloud terminal, server, special terminal that consists of a computer itself, or a device equivalent to a computer. The sensor unit 10B includes an RRI sensor 10 and communication unit 11, and is configured to obtain RRI data from the RRI sensor 10 and transmit the data from the communication unit 11 to the computer 50.

The computer 50 includes chaos index value calculation unit 201, reference value data retention control unit 202, determination target chaos index value calculation unit 203, index value ratio calculation unit 204, determination unit 205, and communication unit 206, which are implemented by the computer. Furthermore, the computer 50 is provided with a storage device 300 and a display device 40. Note that the storage device 300 may exist in the cloud, and the computer 50 may communicate with the storage device 300 to send and receive data in the cloud. The computer 50 obtains the RRI data from the sensor unit 10B via the communication unit 206 and performs the same processing as the brain activity determination apparatus shown in FIG. 3

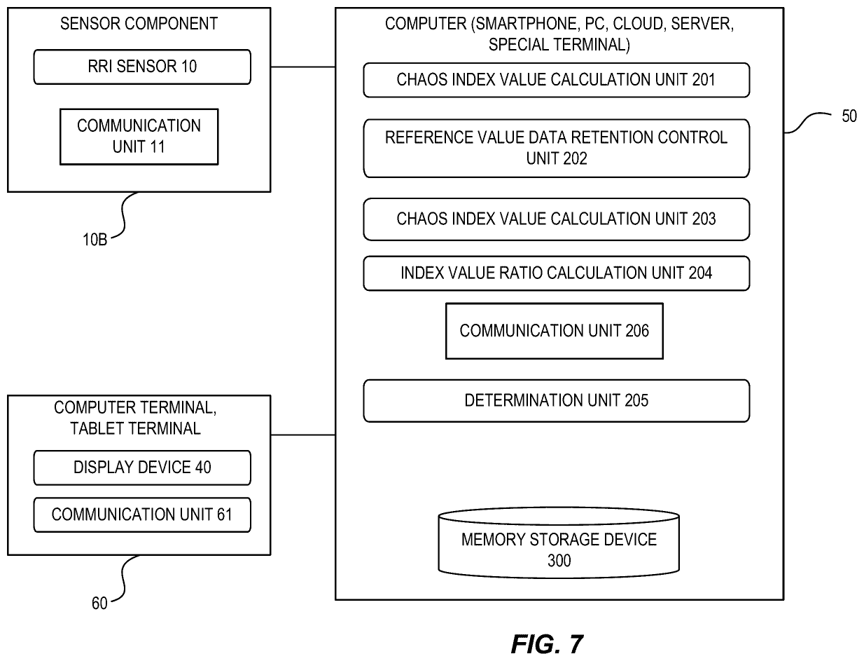

FIG. 7 shows a device configuration diagram of a third embodiment of a brain activity state determination device according to the present disclosure. This embodiment employs substantially the same configuration as that of the brain activity determination apparatus shown in FIG. 6.

The difference in configuration is that a computer terminal (or tablet terminal) 60 with a display device 40 is provided separately from the computer 50. In this embodiment, the computer 50 may have a display device, but the message of the determination result and the information of the average index value ratio AVγ_{A} are displayed on a computer terminal (or tablet terminal) 60 with a display device 40. In this embodiment as well, the storage device 300 may exist in the cloud, and the computer 50 may communicate with the storage device 300 in the cloud to transmit and receive data.

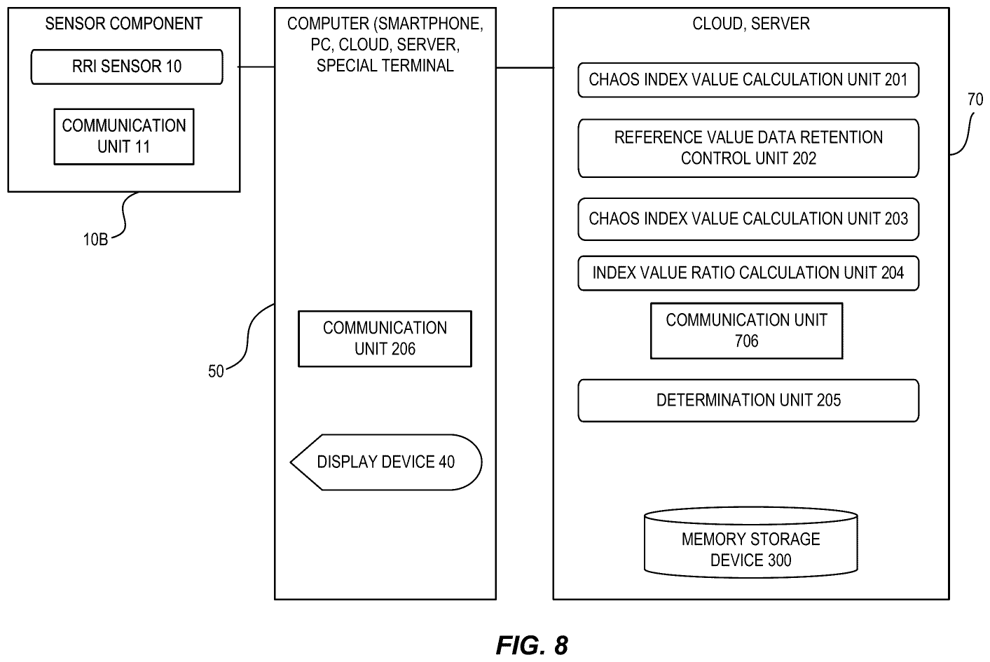

FIG. 8 shows a device configuration diagram of the fourth embodiment of the brain activity state determination device according to the present disclosure. In this embodiment, a configuration in which a cloud computer (or a server computer) 70 is connected to a computer 50 is adopted as the configuration of the brain activity state determination device shown in FIG. 6. The sensor unit 10B is provided with the communication unit 11, the computer 50 is provided with the communication unit 206, the cloud computer (or server computer) 70 is provided with the communication unit 706, and the communication unit 11, the communication unit 206, the communication unit 706 mutually transmit and receive data, etc., to one another and function as a brain activity determination device.

The sensor unit 10B is provided with the RRI sensor 10, acquires RRI data, and transmits it to the cloud computer (or server computer) 70 via the computer 50. The computer 50 is provided with a display device 40, which displays a message of the determination result and the average index value ratio AVγ_{A}. The cloud computer (or server computer) 70 includes computer-implemented chaos index value calculation unit 201, reference value data retention control unit 202, and chaos index value calculation unit 203, index value ratio calculation unit 204, determination unit 205, and communication unit 706. Furthermore, the cloud computer (or server computer) 70 is provided with a storage device 300. Of course, the storage device 300 may not be provided with the cloud computer (or server computer) 70, but may exist in the cloud, and the cloud computer (or server computer) 70 may communicate with the storage device 300 to transmit and receive data in the cloud.

### Embodiment Using Resting State Reference Value Data

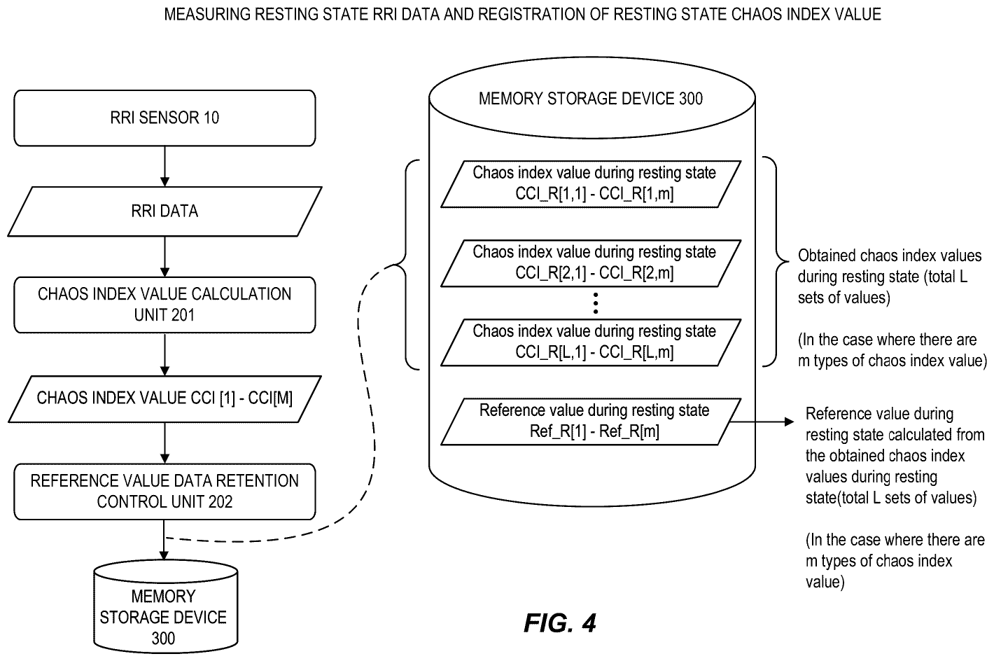

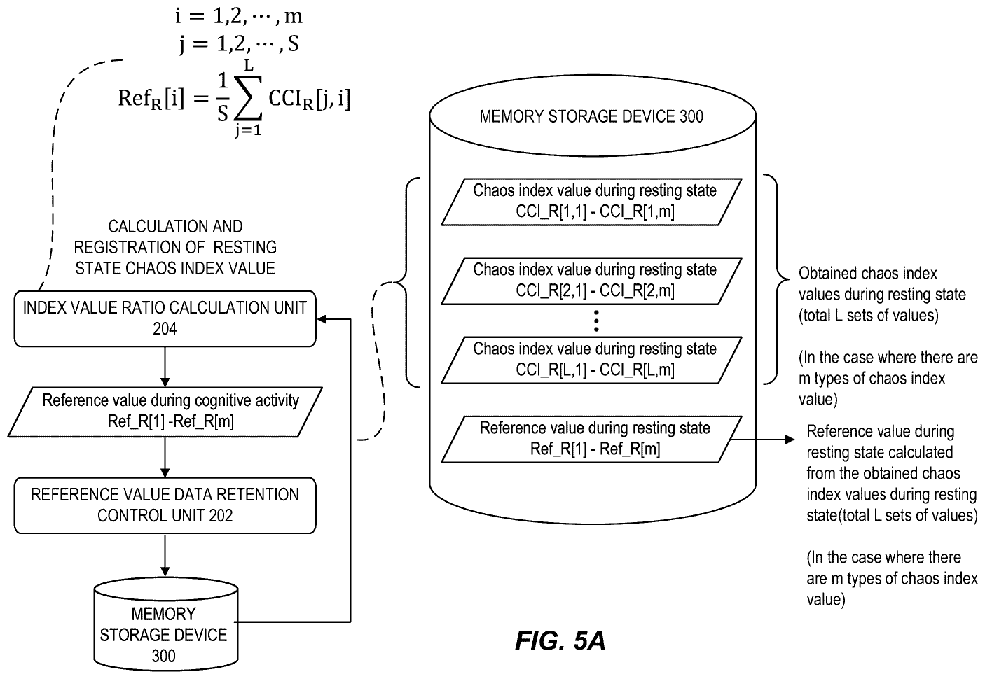

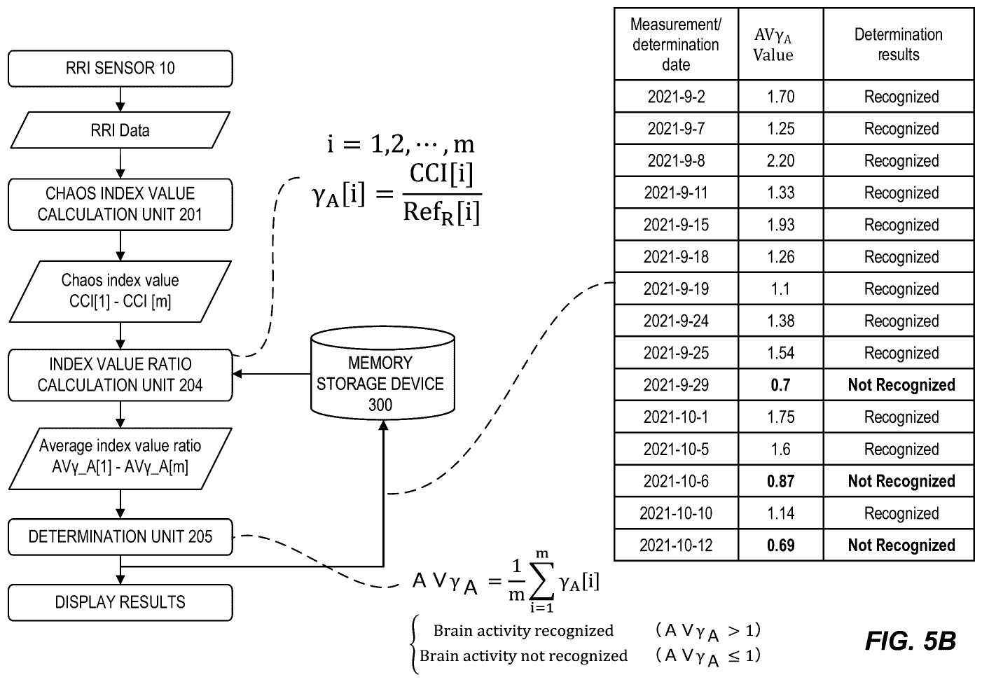

A brain activity determination device according to an embodiment of the present disclosure is a device having any one of the configurations shown in FIGS. 6-8 above. In this embodiment, the reference value data retention control unit 202 stores the outputs obtained by inputting the RRI data obtained from the subject in a resting state (the first state in which the load on the brain obtains the reference value data) to the chaos index value calculation unit 201 in storage device 300 as the resting chaos index value data CCI[1] to CCI[m] (FIG. 4). The resting state may normally be a state in which the subject is lying on a bed, is not physically exhausted, and is not subject to any stress on the brain. It refers to a state in which there is no load on the brain, including the physical load. In this embodiment, the chaos index value calculating unit 201 obtains, for example, m (=9) types of chaos index values in the S time series, and then obtains the average of the S time series for each type of chaos index and calculates and stores the resting reference value data Ref_{R}[1] to Ref_{R}[m] (FIG. 5A).

The determination target chaos index value calculation unit 203 inputs the RRI data obtained from the determination target person in the brain task execution state (the load on the brain in the second state) to the chaos index value calculation unit 201 to obtain a chaos index value to be evaluated. Here, the person to be evaluated is the same person as the subject from whom the standard value data at the time of performing a cognitive activity was obtained. Further, since the chaos index value calculating unit 201 obtains, for example, m (=9) types of chaos index values, m types of CCI[1] to CCI[m], are obtained as determination target chaos index value data. Furthermore, a brain tasks means a task that prompts the activation of the network between brain regions that is most activated in intellectual/cognitive activities called Executive Control Network (ECN) or CEN (Central Executive Network) in the research field of brain networks. For example, it refers to mental arithmetic, puzzles, quizzes (three-choice, four-choice), etc., including those used in the experiments of FIGS. 1 and 2. A plurality of types of brain tasks may be prepared and selected according to the preferences of the operator. Alternatively, the questions may be presented at random regardless of the type, and it is considered that favorable results can be expected in this way.

The index value ratio calculation unit 204 calculates index value ratios γ_{A}[1] to γ_{A}[m] corresponding to the plurality of types (m). The index value ratio calculating unit 204 averages the obtained index value ratios γ_{A}[1] to γ_{A}[m] to obtain an average index value ratio AVγ_{A} (FIG. 5B).

Hence, the average index value ratio AVγ_{A} is obtained by the following formula. Although the arithmetic mean is used in this embodiment, it is a representative value of γ_{A}[1] to γ_{A}[m] and is calculated using γ_{A}[1] to γ_{A}[m] depending on the purpose, and any averaging methods using γ_{A}[1] to γ_{A}[m] are acceptable (for example, geometric mean, mean of logarithms, etc.).

$$i = 1 , 2 , … , m \\ {γ}_{A} [ i ] = \frac{CCI [ i ]}{{Ref}_{R} [ i ]} \\ AV {γ}_{A} = \frac{1}{m} {\sum }_{i = 1}^{m} {γ}_{A} [ i ] \qquad [ No . 1 ]$$

In this embodiment, the brain activity threshold is set to 1, and the determination unit 205 obtains the determination result that
brain activity is recognized (when AVγ_{A}>1) and
brain activity is not recognized (when AVγ_{A}≤1).

The message “brain activity observed” or “brain activity not observed” and the information of the average index value ratio AVγ_{A}, which are the results obtained above, are sent to the display device 40 and displayed. FIG. 5B summarizes the determination results and average index value ratios AVγ_{A} obtained on 15 days between September 2^{nd} and October 12^{th} in a table.

### Embodiment Using Reference Value Data During a Cognitive Activity

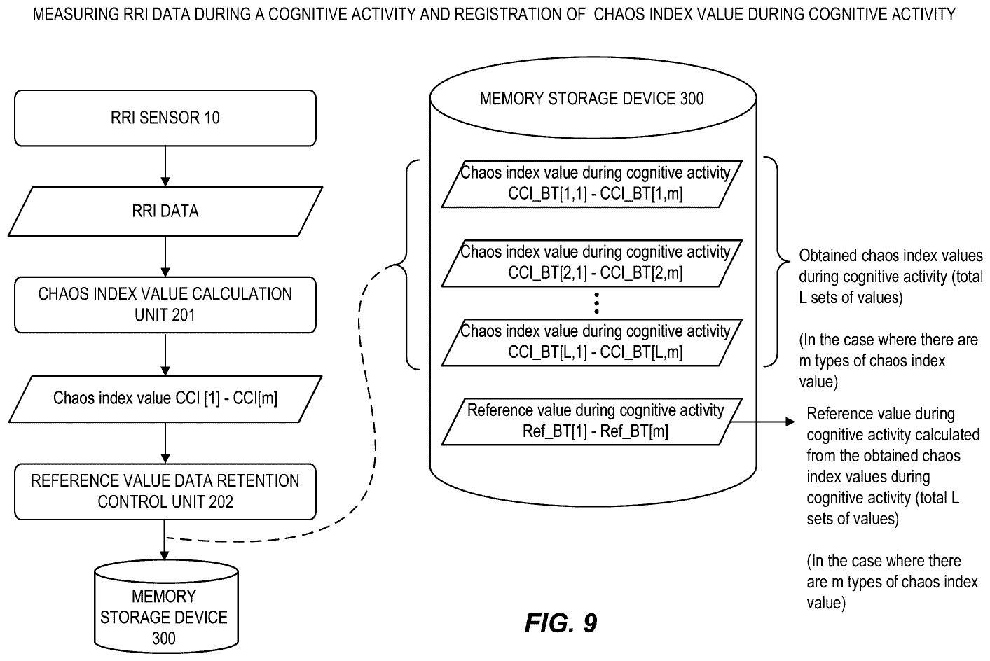

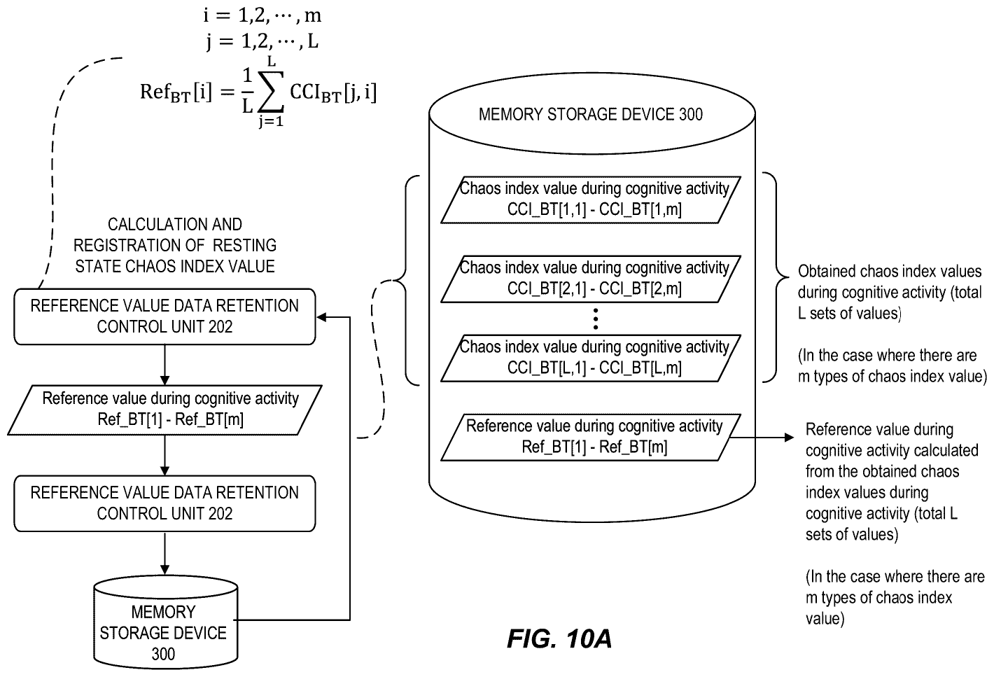

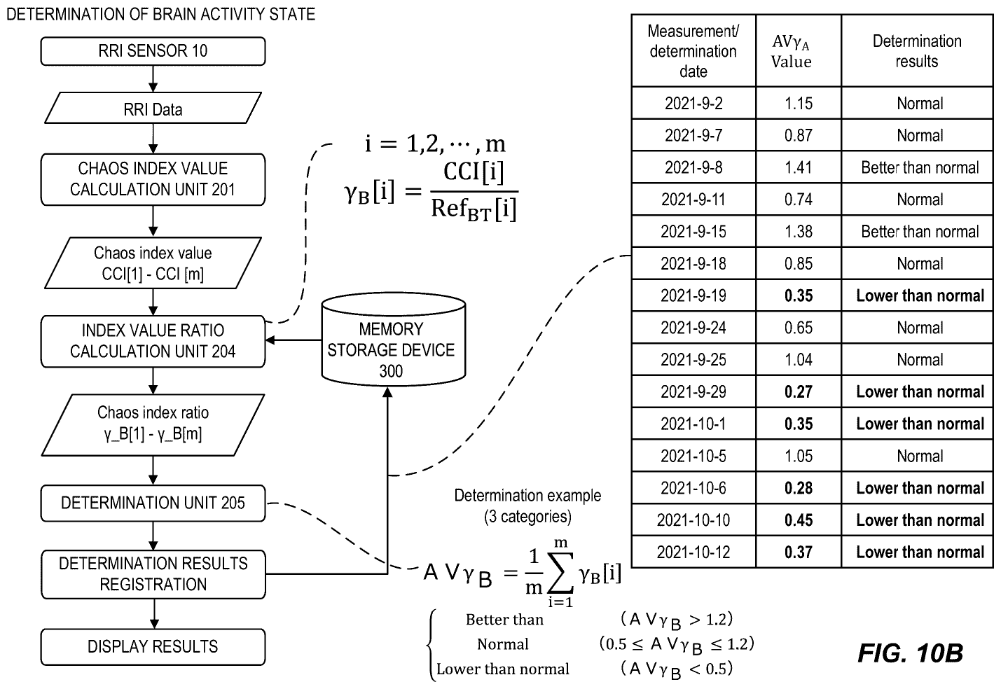

A brain activity determination device according to an embodiment of the present disclosure is a device with any one of the configurations shown in FIGS. 6-8 above. In this embodiment, the reference value data retention control unit 202 stores the output obtained by inputting the RRI data obtained from the subject whose brain is in the cognitive activity state (the load on the brain is in the first state (actually, the third state)) to the chaos index value calculation unit 201 in the storage device 300 as chaos index value data CCI[1] to CCI[m] at the time of performing a cognitive activity (FIG. 9). Here, the term “during a cognitive activity” refers to a state in which the subject is sitting on a chair and performing a mental arithmetic or Sudoku as described with reference to FIGS. 1 and 2. In this embodiment, it is assumed that the chaos index value calculating unit 201 obtains m (=9) types of chaos index values, for example. are obtained for each type, and the cognitive activity reference value data Ref_{BT}[1] to Ref_{BT}[m] are calculated and stored (FIG. 10A).

The determination target chaos index value calculation unit 203 obtains the chaos index value to be evaluated by inputting the RRI data obtained from the determination target person in the brain task execution state (the load on the brain to the second state (actually, the fourth state)) to the calculation unit 201. Here, the person to be evaluated is the same person as the subject who obtained the reference value data during a cognitive activity. Further, since the chaos index value calculating unit 201 obtains m (=9) kinds of chaos index values, for example, m kinds of chaos index value data to be judged, CCI[1] to CCI[m], are to be obtained. Furthermore, the brain task is the same as in the above embodiments using resting state reference value data. Although the brain tasks are the same, in this embodiment, the determination subject whose brain was initially not in the resting state but in the cognitively active state was subject to the brain task execution; therefore, the determination subject was identified as “determination subject in brain task execution state (load on the brain is in the second state (actually, the fourth state))” and not as “determination subject in brain task execution state (load on brain is second state).”

The index value ratio calculating unit 204 calculates the index value ratios γ_{B}[1] to γ_{B}[m] corresponding to the plurality of types (m). The index value ratio calculation unit 204 averages the obtained index value ratios to obtain an average index value ratio AVγ_{B} (FIG. 10B). That is, the average index value ratio AVγ_{B} is obtained by the following formula. Although the arithmetic mean is used in this embodiment, it is a representative value of γ_{B}[1] to γ_{B}[m] and calculated using γ_{B}[1] to γ_{B}[m] depending on the purpose and any averaging methods using γ_{B}[1] to γ_{B}[m] are acceptable (for example, geometric mean, mean of logarithms, etc.).

$$i = 1 , 2 , … , m \\ {γ}_{B} [ i ] = \frac{CCI [ i ]}{{Ref}_{BT} [ i ]} \\ AV {γ}_{B} = \frac{1}{m} {\sum }_{i = 1}^{m} {γ}_{B} [ i ] \qquad [ No . 2 ]$$

In this embodiment, the brain activity thresholds are set to 0.5 and 1.2, and the determination unit 205 performs the following three steps:
Better than normal (when AVγ_{B}>1.2)
Normal (0.55 AVγ_{B}≤1.2)
A determination result lower than normal (when AVγ_{B}<0.5)
   Here, the threshold is a reference value at the present time, and may be changed in consideration of the situation after the implementation of the present disclosure. However, the definitions that do not change are as follows:
Better than normal must be a value greater than 1.
Lower than normal must be less than 1.
Normal should be an intermediate value between the above two.

The message “better than normal,” “normal,” or “lower than normal,” which is the determination result obtained above, and the information of the average index value ratio AVγ_{B} are sent to the display device 40 and displayed. FIG. 10B summarizes the determination results and average index value ratios AVγ_{B} obtained on 15 days between September 2^{nd} and October 12^{th} in a table.

### Embodiment α for Determining Chronic Brain Fatigue Using Long-Term Processing Results Using Reference Value Data Generated During a Cognitive Activity

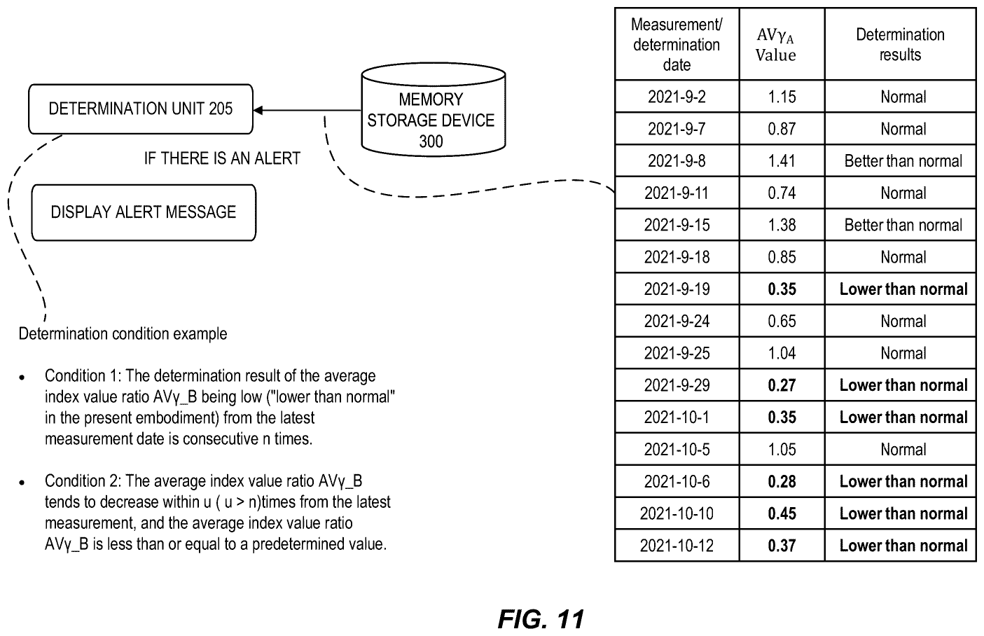

The brain activity determination apparatus according to the Embodiment α of the present disclosure has any one of the configurations shown in FIGS. 6-8 above. In the present Embodiment α, data processing is performed using reference value data generated during a cognitive activity. “Long-term” mean that sufficient determination results have been obtained for determination of chronic brain fatigue, for example, determination results for one month or more are sufficient. FIG. 11 shows an example of long-term processing results according to the embodiment using reference value data generated during a cognitive activity. In this example, it is assumed that 15 determination results for about one month are stored in the storage device 300 (the table shown on the right side of FIG. 11).

In this Embodiment α, the determination unit 205 determines the following conditions.

Condition 1: The determination result of the average index value ratio AVγ_{B} being low (“lower than normal” in the present Embodiment α) from the latest measurement date is consecutive n times.

Condition 2: The average index value ratio AVγ_{B} tends to decrease within u times from the latest measurement, and the average index value ratio AVγ_{B} within v (<u) times is less than or equal to a predetermined value.

Note that n, u, and v are positive integers and can be determined as appropriate. In this way, the determining unit 205 constitutes a first brain fatigue determining unit for determining chronic brain fatigue based on the stored determination results and the decreasing tendency of the average index value ratio at that time.

When at least one of the above Conditions 1 and 2 (or both) is satisfied, a warning message to the effect that the state of chronic brain fatigue and the content information of Conditions 1 and 2 are sent to the display device 40. Not only can it be displayed and make the user aware, but it can also be transmitted from the communication unit 206 (706) of the present Embodiment α to a mobile terminal or the like other than the brain activity state determination device and displayed on the display device.

Brain fatigue is caused by first, constant sleep deprivation, second, constant fatigue, third, depression and mental illness, and fourth, brain inactivity due to external factors (noise, discomfort, anxiety, etc.). Then, It is of great significance that the person who owns the device for determining the state of brain activity and the manager such as a superior are aware of this condition.

### Embodiment β: Using Resting State Reference Value Data and Reference Value Data Generated During a Cognitive Activity

The brain activity determination apparatus according to the Embodiment β of the present disclosure has any one of the configurations shown in FIGS. 6-8 above. In the present Embodiment β, the reference value data retention control unit 202 stores the output obtained by inputting the RRI data obtained from a subject in a resting state (first state of load on the brain) to the chaos index value calculation unit 201 in the memory storage device 300 as the resting chaos index data (FIG. 4).

In the present Embodiment β, the reference value data retention control unit 202 stores the output obtained by inputting the RRI data obtained from subjects in which the brain is in a cognitive activity state (the load on the brain is in the first state (actually, the third state)) to the chaos index value calculation unit 201 in the memory storage device 300 as the chaos index value data during cognitive activity (FIG. 9). Here, the term “during a cognitive activity” refers to a state in which the subject is sitting on a chair and performing a mental arithmetic or Sudoku as described with reference to FIGS. 1 and 2. In the present Embodiment β, the chaos index value calculation unit 201 obtains, for example, m (=9) types of chaos index values in the L time series, and then obtains the average of the L time series for each type of chaos index and calculates and stores the cognitive activity reference value data Ref_{BT}[1] to Ref_{BT}[m] (FIG. 10A).

The determination target chaos index value calculation unit 203 inputs the RRI data obtained from the determination target person in the brain task execution state (the load on the brain in the second state (actually, the fourth state)) to the above mentioned chaos index value calculation unit 201 to obtain a chaos index value to be evaluated. Here, the person to be evaluated is the same person as the subject from whom the standard value data at the time of performing a cognitive activity was obtained. Further, since the chaos index value calculating unit 201 obtains, for example, m (=9) types of chaos index values, m types of CCI[1] to CCI[m], are obtained as determination target chaos index value data. Furthermore, the brain task is set to be the same as in the embodiments using resting state reference data. Measurement is started at 0:00:00 (hour:minute:second), and the measurement is performed n times (in this embodiment, nine times as an example) at intervals of 10 seconds for five minutes.

$$\begin{matrix}\cdot & 0 : 00 : 00 - 0 : 05 : 00 First time \\ \cdot & 0 : 00 : 10 - 0 : 05 : 10 Second time \\ \cdot & 0 : 00 : 20 - 0 : 05 : 20 Third time \\ \cdot & 0 : 01 : 20 - 0 : 06 : 20 n ( = 9 ) th time\end{matrix}$$

As described above, n×m kinds of determination target chaos index value data CCI[1][1] to CCI[n][m] are obtained. The index value ratio calculation unit 204 divides the determination target chaos index value data CCI[1][1] to CCI[n][m] by the reference value data at rest to calculate resting index value ratios γ_{A}[1][1] to γ_{A}[n][m], and the cognitive activity index value ratio γ_{B}[1][m] are obtained by dividing the determination target chaos index value data CCI[1][1] to CCI[n][m] by the cognitive activity reference value data γ_{B}[1][1] to γ_{B}[n][m], and the respective average index value ratios AVγ_{A}[1] to AVγ_{A}[n] and AVγ_{B}[1] to AVγ_{B}[n] are then calculated.

Regarding the average index value ratios AVγ_{A}[1] to AVγ_{A}[n] and AVγ_{B}[1] to AVγ_{B}[n], in this embodiment, the determination unit 205 sets the brain activity thresholds to 0.5 and 1.2 and obtains determination realists based on whether any of the 4 states below applies:
State 1 AVγ_{A}[n]>1 and AVγ_{B}[n]>1.2: Good Brain Activity
State 2 AVγ_{A}[n]>1 and 0.5 s AVγ_{B}[n]≤1.2: Normal Brain Activity
State 3 AVγ_{A}[n]>1 and AVγ_{B}[n]<0.5: Decreased Brain Activity
State 4 AVγ_{A}[n]≤1: Physical Load Present

The determination results from above can be obtained, one in every 10 seconds, n (=9) in total. If State 3 is continuous over 4 data points up to the latest n (=9)th or if State 4 is over 4 data points up to the latest n (=9)th, then “Decreased Brain Activity” or “Physical Load Present” messages respectively as well as the average index value ratios AVγ_{A}[n] and AVγ_{B}[n] get set by the display device 40 and displayed on the panel. This not only makes the user aware of the condition, but also the information can be transmitted from the communication unit 206 (706) of the present Embodiment β to a portable terminal or the like other than the brain activity state determination device and displayed on the display device.

Thus, in this embodiment, the above mentioned index value ratio calculation unit 204 calculates the above mentioned resting state reference data, the rest index value ratio, which is the ratio to the chaos index value to be determined, as well as cognitive activity index value ratio, which is the ratio of the reference value data at the time of performing a cognitive activity and the chaos index value to be determined. In addition, the determination unit 205 determines the subject's brain activity state by comparing the brain activity threshold corresponding to the resting state with the resting index value ratio, and comparing the brain activity threshold corresponding to the cognitive activity with the cognitive activity index value ratio to determine the brain activity state.

An Embodiment of Accumulating Information on Determination Results N (Pieces) and Average Index Value Ratios Avγ_{A}[N] and Avγ_{B}[N] Over a Long Period of Time in Embodiment β, and Determining Chronic Brain Fatigue Using this Accumulated Data Using the Method of Embodiment α

A brain activity determination device according to an embodiment of the present disclosure is a device having any one of the configurations shown in FIGS. 6-8 above. In this example, it is assumed that the storage device 300 stores information on n (=9) determination results and average index value ratios AVγ_{A}[n] and AVγ_{B}[n] over 15 days of one month.

In the above mentioned Embodiment β, real-time estimation (01 second interval) was performed using data obtained by dividing the run time from 0:00:00 to 0:06:20 into n (=9) segments, but unlike this embodiment, this embodiment accumulates the data measured in real time for 15 days to measure chronic brain fatigue. That is, real-time measurement data are accumulated over a long period of several days, and are used to measure chronic brain fatigue.

In this embodiment, the determination unit 205 obtains a determination result based on which of the following four states the brain activity threshold is 0.5 and 1.2:
State 1 AVγ_{A}[n]>1 and AVγ_{B}[n]>1.2: Good Brain Activity
State 2 AVγ_{A}[n]>1 and 0.5≤AVγ_{B}[n]≤1.2: Normal Brain Activity
State 3 AVγ_{A} [n]>1 and AVγ_{B}[n]<0.5: Decreased Brain Activity
State 4 AVγ_{A}[n]≤1: Physical Load Present

The determination results from above can be obtained, one in every 10 seconds, n (=9) in total per day. If State 3 is continuous over 4 data points or more up to the latest n (=9)th, the determination results of “Decreased Brain Activity Level 3” will be obtained. If State 3 is continuous over 5 data points, for example, the determination results of “Decreased Brain Activity Level 2” will be obtained. If State 3 is continuous over 3 or 4 data points, for example, the determination results of “Decreased Brain Activity Level 1” will be obtained. Otherwise, it is considered that no decrease in brain activity is observed. In this embodiment, the determination unit 205 determines the following conditions. Condition 1: “Brain Activity Level Decreased” continues n times from the latest measurement date. Condition 2: within u (u>n)th times from the latest measurement time, the added value of the “Brain Activity Level Decrease” numerical value is equal to or greater than a predetermined value. Note that n and u are positive integers and can be determined as appropriate.

When at least one of the above Conditions 1 and 2 (or both) is satisfied, an alarm message to the effect that the subject is in a state of chronic brain fatigue and the content information of Conditions 1 and 2 are sent to the display device 40. Not only can the information be displayed and the user can be made aware, but it can also be transmitted from the communication unit 206 (706) of this embodiment to a portable terminal or the like other than the brain activity state determination apparatus and displayed on the display device.

While several embodiments of the disclosure have been described, these embodiments are provided by way of example and are not intended to limit the scope of the invention. These novel embodiments can be implemented in various other forms, and various omissions, replacements, and modifications can be made without departing from the scope of the invention. These embodiments and their modifications are included in the scope and gist of the invention, and are included in the scope of the invention described in the claims and equivalents thereof.

### DESCRIPTIONS OF REFERENCE NUMBERS

10 Sensor
10B Sensor unit
11 Communication unit
20 Smart watch
40 Display device
50 Computer
201 Chaos index value calculation unit
202 Reference value data retention control unit
203 Determination target chaos index value calculation unit
204 Index value ratio calculation unit
205 Determination unit
206 Communication unit
300 Storage device
706 Communication unit

1. A brain activity determination device comprising:
a chaos index value calculation unit for calculating a chaos index value which is an index for determining a chaotic nature of data in chronological order;
a reference value data retention control unit that retains an output obtained by inputting RRI data obtained from a subject placed in a first state for obtaining reference data to the chaos index value calculation unit as reference value data in a storage device;
a determination target chaos index value calculation unit for obtaining the chaos index value to be evaluated by inputting the RRI data obtained from the subject in a second state for obtaining evaluation target data, to the chaos index value calculation unit;
an index value ratio calculation unit for calculating the index value ratio, which is the ratio between the reference value data and the chaos index value to be evaluated; and
a determination unit for determining the brain activity state of the subject based on a comparison of the brain activity threshold and the index value ratio.

2. The brain activity determination device according to claim 1, wherein:
the chaos index value calculation unit calculates a plurality of types of chaos index values;
the reference value data retention control unit retains reference value data corresponding to the plurality of types in the storage device;
the determination target chaos index value calculation unit obtains determination target chaos index values corresponding to the plurality of types; and
the index value ratio calculation unit calculates index value ratios corresponding to the plurality of types.

3. The brain activity determination device according to claim 2, wherein the index value ratio calculation unit calculates index value ratios corresponding to the plurality of types, and averages the obtained index value ratios to obtain an average index value ratio.

4. The brain activity determination device according to claim 1, wherein the reference value data retention control unit stores an output obtained by providing RRI data obtained from a subject in a resting state to the chaos index value calculation unit as resting state reference value data.

5. The brain activity determination device according to claim 1, wherein RRI data obtained from a subject whose brain is in a cognitive activity state is given to the chaos index value calculation unit and the obtained output is stored by the reference value data retention control unit stores as reference value data during cognitive activity.

6. The brain activity determination device according to claim 2, wherein:
the index value ratio calculation unit calculates the index value ratios corresponding to the plurality of types, and averages the obtained index value ratios to obtain an average index value ratio;
the determination unit stores the average index value at the time of storing step as a result of performing determination steps at least five times; and
further comprising a first brain fatigue determination unit for determining chronic brain fatigue based on the stored determination result and a decreasing tendency of the average index value ratio at that time.

7. The brain activity determination device according to claim 1, wherein:
the reference value data retention control unit:
inputs the RRI data obtained from the subject in a resting state to the chaos index value calculating unit and retains the output obtained as reference value data at rest, and
inputs the RRI data obtained from the subject whose brain is in a state of cognitive activity to the chaos index value calculation unit and storing the output obtained as reference value data during cognitive activity in a storage device;
the index value ratio calculating unit calculates:
a resting index value ratio, which is a ratio of the reference value data at rest and the chaos index value to be evaluated, and
a cognitive activity index value ratio, which is a ratio of the cognitive activity reference value data to the determination target chaos index value; and
the determination unit compares a brain activity threshold corresponding to a resting state with the resting index value ratio and compares a brain activity threshold corresponding to cognitive activity with the cognitive activity index value ratio to determine the brain activity state.

8. The brain activity determination device according to claim 7, wherein
the index value ratio calculation unit calculates a plurality of rest index value ratios and cognitive activity index value ratios at a plurality of time intervals using RRI data obtained in a predetermined time interval;
and the determination unit determines the brain activity state of the determination subject based on comparison with a plurality of types of brain activity thresholds respectively set for a plurality of rest index value ratios and cognitive activity index value ratios.

9. The brain activity determination device according to claim 8, wherein:
the index value ratio calculation unit calculates the index value ratios corresponding to the plurality of types, and averages the obtained index value ratios to obtain an average index value ratio;
the determination unit stores the average resting state index ratio and average cognitive activity index ratio at the time of storing step as a result of performing determination steps at least five times; and
further comprising a second brain fatigue determination unit for determining chronic brain fatigue based on the stored determination result and the decreasing tendency of the average rest index value ratio and the average cognitive activity index value ratio at the time of storing data.

10. A computer-readable media storing instructions that, when executed by a computer, cause it to act as a brain activity determination device comprising:
a chaos index value calculation unit for calculating a chaos index value that is an index for determining a chaotic nature of data in chronological order;
a reference value data retention control unit that retains an output obtained by inputting RRI data obtained from a subject placed in a first state, which is the state for obtaining the reference data, to the chaos index value calculation unit as reference value data in a storage device;
a determination target chaos index value calculation unit for obtaining the chaos index value to be evaluated, which is the data to be evaluated, by inputting the RRI data obtained from the subject to be evaluated whose brain load is considered to be in a second state in which evaluation target data is to be obtained, to the chaos index value calculation unit;
an index value ratio calculation unit for calculating an index value ratio that is a ratio between the reference value data and the chaos index value to be evaluated; and
a determination unit for determining the brain activity state of the determination subject based on a comparison between the brain activity threshold and the index value ratio to determine the brain activity state.

11. The computer-readable media according to claim 10, wherein:
the chaos index value calculating unit calculates a plurality of types of chaos index values;
the reference value data retention control unit retains the reference value data corresponding to the plurality of types;
the determination target chaos index value calculating unit obtains determination target chaos index values corresponding to the plurality of types; and
the index value ratio calculation unit calculates the index value ratios corresponding to the plurality of types.

12. The computer-readable media according to claim 11, wherein the index value ratio calculating unit calculates index value ratios corresponding to the plurality of types and averages the obtained index value ratios to obtain an average index value ratio.

13. The computer-readable media according to claim 10, wherein the reference value data retention control unit provides an output obtained by inputting the RRI data obtained from the subject in the resting state to the chaos index value calculation unit as resting reference value data.

14. The computer-readable media according to claim 10, wherein the reference value data retention control unit provides an output obtained by inputting the RRI data to the chaos index value calculating unit as cognitive activity baseline data.

15. The computer-readable media according to claim 11, wherein:
the index value ratio calculation unit calculates index value ratios corresponding to the plurality of types and obtains an average index value ratio by averaging the obtained index value ratios; and
the determination unit performs determination steps at least five times and stores the determination result and the average index value ratio at the time of storing data; and
further comprising first brain fatigue determination unit determining chronic brain fatigue based on the stored determination result and the declining trend of the average index value ratio at the time of storing data.

16. The computer-readable media according to claim 10, wherein:
the reference value data retention control unit:
stores an output obtained by giving the RRI data obtained from the subject in the resting state to the chaos index value calculation unit as resting reference value data in the storage device, and
stores output obtained by inputting RRI data obtained from subjects whose brain is in a cognitively active state to the chaos index value calculation unit in a storage device as reference value data during cognitive activity;
the index value ratio calculation unit calculates the resting index value ratio, which is the ratio of the reference value data at rest and the chaos index value to be evaluated and the cognitive activity index value ratio, which is a ratio of the reference value data at the time of cognitive activity and the chaos index value to be determined; and
the determination unit:
determines the comparison between brain activity threshold corresponding to a resting state and the resting index value ratio to determine a brain activity state, and
determines the brain activity state of the determination subject based on a comparison between the brain activity threshold corresponding to cognitive activity and the cognitive activity index value ratio.

17. The computer-readable media according to claim 16, wherein:
the index value ratio calculation unit calculates multiple resting index ratios and cognitive activity index ratios at multiple time intervals, using RRI data obtained at constant time intervals; and
the determination unit determines the brain activity state of the determination subject based on a comparison with multiple types of brain activity thresholds set for multiple resting index ratios and cognitive activity index ratios.

18. The computer-readable media according to claim 17, wherein:
the index value ratio calculation unit calculates the index value ratio corresponding to the above multiple types and then averages the index value ratios to get the average index value ratio;
the determination unit performs determination steps at least five times and stores the determination result and the average resting index ratio and average cognitive activity index ratio at the time of storing data; and
further comprising a second brain fatigue determination unit for determining chronic brain fatigue based on the stored determination result and the declining trend of the average index value ratio at the time of storing data.
